(19) United States
(12) Patent Application Publication
(10) Pub. No.: US 2017/0367608 A1
(43) Pub. Date: Dec. 28, 2017

(54) PERSONAL BRAIN STRUCTURE DISPLAYING DEVICE HAVING INTRACRANIAL ELECTRODES AND ITS DISPLAYING METHOD

(71) Applicant: CHUNG SHAN MEDICAL UNIVERSITY HOSPITAL, Taichung City (TW)

(72) Inventors: YUE-LOONG HSIN, Taichung City (TW); SYU-JYUN PENG, Taichung City (TW)

(21) Appl. No.: 15/406,625

(22) Filed: Jan. 13, 2017

(30) Foreign Application Priority Data
Jun. 28, 2016 (TW) 105120252

(51) Int. Cl. A61B 5/0478; A61B 6/03; A61B 5/00; A61B 5/055; A61N 1/05; A61B 6/00

(52) U.S. Cl. CPC A61B 5/0478; A61N 1/0529; A61B 6/501; A61B 6/461; A61B 5/0042; A61B 5/742; A61B 5/6848; A61B 5/055; A61B 6/032

(57) ABSTRACT

An electrode module is positioned inside an intracranial portion of a human head. Then, it captures brain images of the human head so multiples two dimensional (2D) cross-sectional images are obtained. The electrodes can be seen in one or more 2D cross-sectional images. A brain functional map adjusting portion is provided to obtain the 2D cross-sectional images and then to conduct a proportional deformation process for the images in the brain functional map database. By combining the processed images in the brain functional map database and the 2D cross-sectional images, multiple combined cross-sectional images can be obtained for display. So, the effects of intracranial electrodes are better than the traditional way. In addition, the brain structure information of a patient contains the precise positions of the electrodes and the corresponding brain functional areas.

### BACKGROUND OF THE INVENTION

### 1. Field of Invention

This invention relates to a personal brain structure displaying device having intracranial electrodes and its displaying method. In which, the effects of intracranial electrodes are better than the traditional way. Plus, the brain structure information of a patient contains the precise positions of the electrodes and the corresponding brain functional areas.

### 2. Description of the Prior Art

With regard to a traditional epilepsy surgery, its preparing procedure can be described as follows.

For example, one patient with epilepsy has vigorous shaking or seizures on his or her right hand. Under such condition, we can predict some area of the patient's brain that controlling the right hand is abnormal or injured (could be blood clot, tumor, vascular necrosis, etc.).

First, the medical personnel will conduct a brain scan by using the scanning computed tomography or related technology. So, the information about the brain structure can be obtained. After which, based on the medical personnel's experience, the brain structure can be divided into several portions with specific functions.

The medical personnel will place several electrodes on the scalp where is corresponding to the specific portion of the brain for controlling right hand. For example, a set of nine electrodes (such as a 3×3 distributed type) can be stuck on the scalp for collecting all the electric signal variations. When the epilepsy is started, the electric variation information collected from all these electrodes is very useful to determine which electrode or electrodes have the strongest electric signal. The area will be considered as the staring point (or zone) for the epilepsy. However, it is possible to apply electricity to certain area of the brain via the electrode or electrodes and then to observe the corresponding reaction of this patient with epilepsy. Then, it can be known which electrode or electrodes might activate the right hand's vigorous shaking. Therefore, the medical personnel will know that the area is highly possible to cause the abnormal function of the brain.

Then, the medical personnel can conduct a surgery to open the skull and find out the corresponding area (the abnormal area) inside the brain so as to check there is any abnormal condition (blood clot, tumor, or vascular necrosis, etc.) or not.

However, the traditional preparing procedure of a traditional epilepsy surgery still has the following disadvantages:
[1] The electrodes recording or stimulation effects are poor. Because these electrodes are disposed outside the skull and scalp, it cannot directly detect the specific area of the brain inside the skull. In addition, if the medical personnel would like to simulate the brain, the medical personnel only can conduct an indirect stimulation to the brain (via the skull and scalp). Thus, the recording or stimulation effects are poor.
[2] The functional areas of a patient's brain cannot be shown easily. When one medical personnel or doctor wants to understand the brain structure of a patient, this medical doctor only can see a lot of two dimensional cross-sectional images via the computed tomography (briefly called CT) or other scanning technology on the screen. But, the medical doctor cannot know the exact position of a specific functional area of brain from the CT scan result. The medical doctor only can rely on his or her personal experience to know the exact position of a specific functional area of this patient's brain. Therefore, it is very inconvenient.
[3] The position of the electrodes and the position of the functional area of brain cannot be combined in one screen. The traditional CT scan result does not show the exact position of functional areas of brain. We cannot know which function corresponds to the position of the electrode from the CT scan result. Hence, the position of the electrodes and the functional areas of brain cannot be combined in one screen.
[4] The brain function map (or brain atlases) cannot be applied on. The traditional brain function map contains many different functional areas in three-dimensional condition. This kind of brain function map is established on the averaged result of many persons by statistical methods. However, every person's brain size and shape are different to another one's. Practically, we cannot apply the existing brain function map on the CT scan result of a specific patient directly. One medical personnel only can predict (based on experience) where the possible position (or boundary) of a specific brain function could be. Thus, it is lack of related auxiliary displaying technique to overcome this problem.

### SUMMARY OF THE INVENTION

The object of this invention is to provide a personal brain structure displaying device having intracranial electrodes and its displaying method. In which, the effects of intracranial electrodes are better than the traditional way. In addition, the brain structure information of a patient contains the precise positions of the electrodes and the corresponding brain functional areas. Particularly, this invention can solve the problems of the traditional one listed as follows. The electrodes recording or stimulation effect are poor. The functional areas of brain a patient cannot be shown. The position of the electrodes and the position of the functional area of brain cannot be combined in one screen. In addition, the brain function map (or brain atlases) is cannot be applied on.

A personal brain structure displaying device having intracranial electrodes comprising:
an electrode module positioned inside a human head, the electrode module having multiple electrodes;
an image capturing module for capturing a brain area image of the human head, the image capturing module being able to obtain a three-dimensional (3D) brain information which includes a plurality of two-dimensional (2D) cross-sectional images; each 2D cross-sectional image including a brain profile line and an inner brain area; at least one 2D cross-sectional image containing an electrode image that is positioned on one of or both of the brain profile line or the inner brain area;
a controller connecting with the electrode module and the image capturing module for obtaining the three-dimensional (3D) brain information;
a brain functional map adjusting portion connecting with the controller, the brain functional map adjusting portion containing a brain functional map database and being able to obtain the 2D cross-sectional images and then to conduct a proportional deformation process so that the brain functional map database matches with corresponding 2D cross-sectional images; a plurality of two-dimensional (2D) adjusted brain functional map cross-sectional images being obtained; each 2D adjusted brain functional map cross-sectional image containing a database brain profile line and several database brain functional zones; during the proportional deformation process, each database brain profile line being proportionally deformed to match with corresponding brain profile line and the brain functional zones being proportionally deformed accordingly to match with and fitted into the 2D cross-sectional images so as to obtain a plurality of combined cross-sectional images that can be transmitted to the controller; and
a displaying portion connecting with the controller for showing out these combined cross-sectional images.

A displaying method of personal brain structure displaying device having intracranial electrodes mainly comprising:
preparing step;
brain image capturing step;
obtaining three-dimensional brain with electrodes information step;
brain functional map adjusting step; and
combining and showing step.

### BRIEF DESCRIPTION OF THE DRAWINGS

FIG. 1. is a perspective view of an application of this invention;

FIG. 2 is the first preferred embodiment of the electrode module of this invention;

FIG. 3 is block diagram of this invention;

FIG. 4 is a view illustrating one example of a portion of the brain scanning procedures;

FIGS. 5A, 5B and 5C are the 2D cross-sectional images taken along the line of VA-VA, VB-VB, and VC-VC respectively;

FIG. 6 shows a view of this invention having related adjusting and combining processes;

FIG. 7 is a view showing one of the combined cross-sectional images;

FIG. 8 is an enlarged view of a selected portion in FIG. 7;

FIG. 9 is the second preferred embodiment of the electrode module of this invention;

FIG. 10A is the 2D cross-sectional images taken along the line of XA-XA;

FIG. 10B is the 2D cross-sectional images taken along the line of XB-XB;

FIG. 10C is the 2D cross-sectional images taken along the line of XC-XC;

FIG. 11 is the third preferred embodiment of the electrode module of this invention;

FIG. 12 is an enlarged view of a selected portion in FIG. 11;

FIG. 13 is a view when it is viewed from another angle;

FIG. 14A is a simplified cross-sectional view taken along the line of XIVA-XIVA;

FIG. 14B is a simplified cross-sectional view taken along the line of XIVB-XIVB;

FIG. 15 is a flow chart of this invention.

### DESCRIPTION OF THE PREFERRED EMBODIMENT

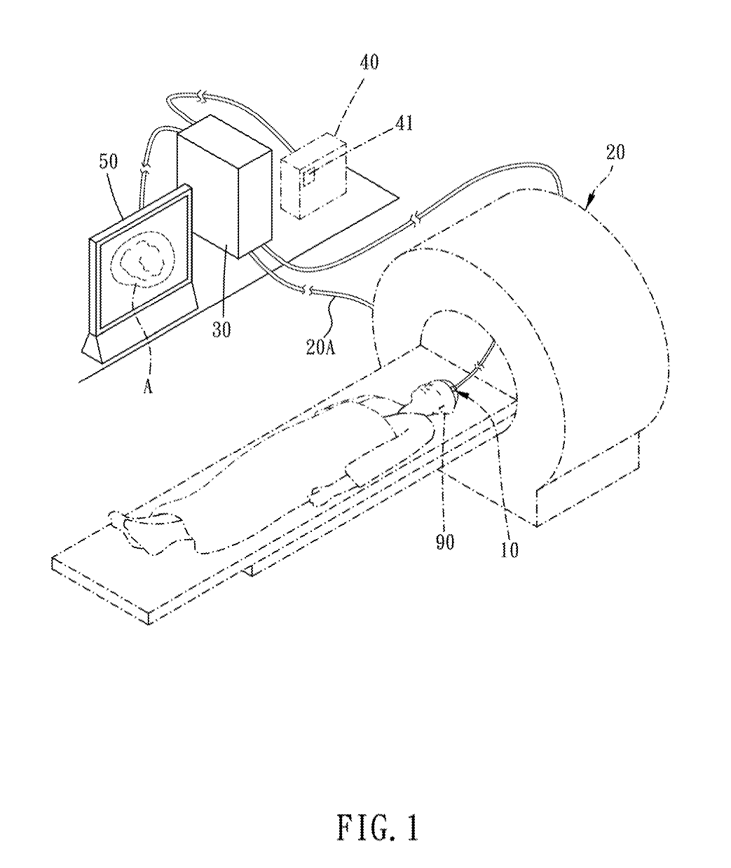

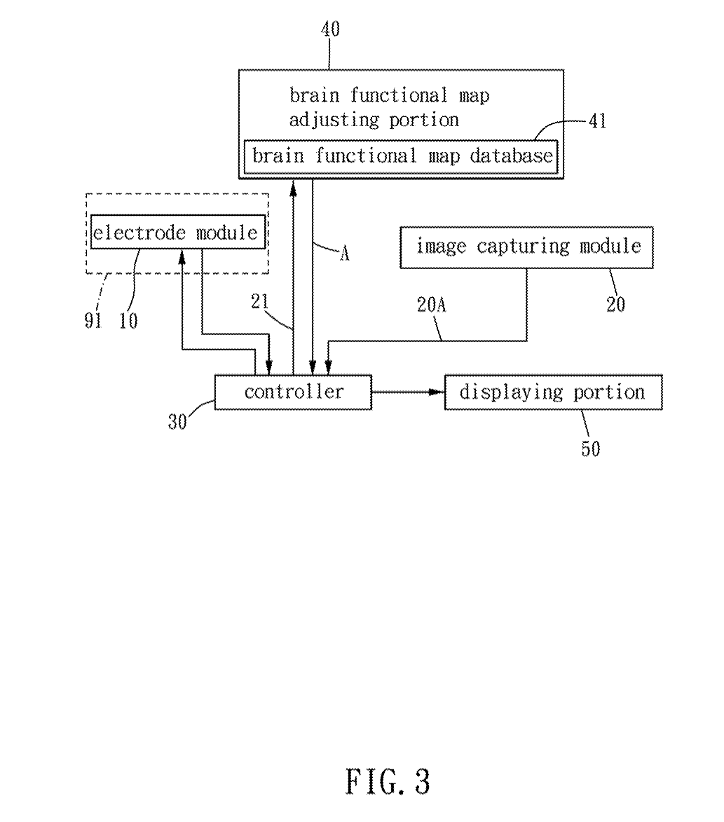

Referring to FIGS. 1, 2, and 3, this invention relates to a personal brain structure displaying device having intracranial electrodes and its displaying method. The device of this invention, it mainly includes an electrode module 10, an image capturing module 20, a controller 30, a brain functional map adjusting portion 40, and a displaying portion 50.

With regard to this electrode module 10, it is positioned inside an intracranial portion 91 of a human head 90. The electrode module 10 has multiple electrodes 11.

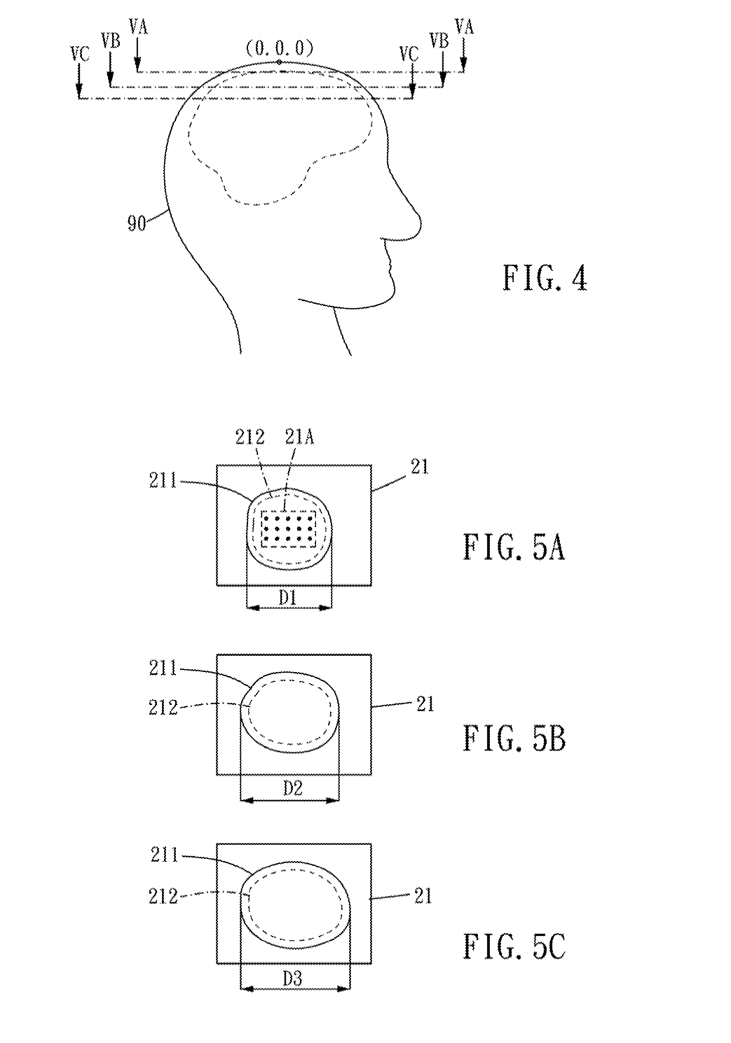

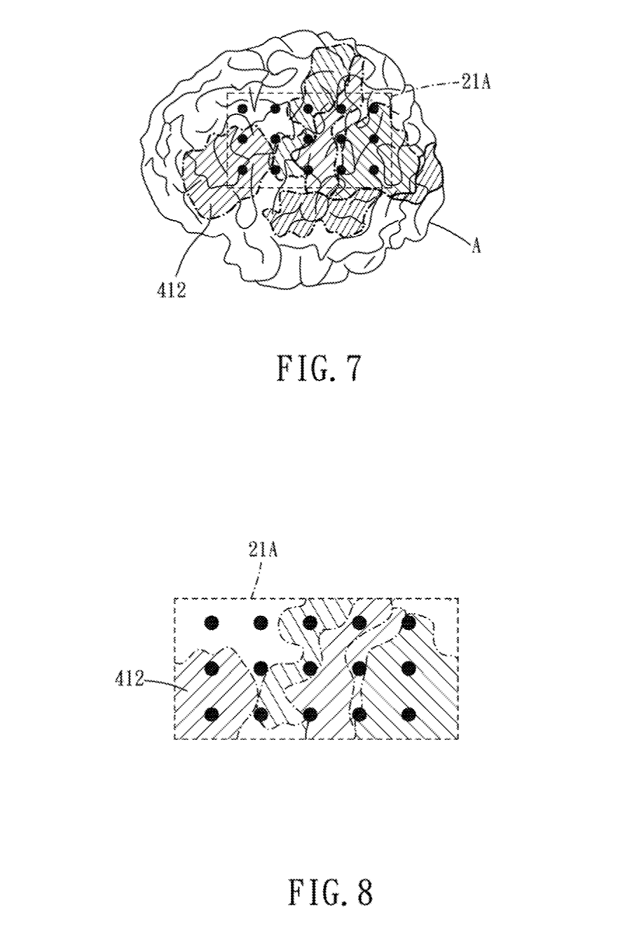

About the image capturing module 20, it is provided for capturing a brain area image (referring to FIG. 4) of the human head 90. This image capturing module 20 is able to obtain a three-dimensional (3D) brain information 20A which includes a plurality of two dimensional (2D) cross-sectional images 21 (illustrated as FIGS. 5A, 5B, and 5C; having a first image width D1, a second image width D2, and a third image width D3 respectively). Each 2D cross-sectional image 21 includes a brain profile line 211 and an inner brain area 212. At least one 2D cross-sectional image 21 contains an electrode image 21A (referring to FIG. 7) that is positioned on one of or both of the brain profile line 211 and the inner brain area 212. Furthermore, the brain profile line 211 just means the outline of the shape of the inner brain area 212.

The controller 30 connects with the electrode module 10 and the image capturing module 20 for obtaining the three-dimensional (3D) brain information 20A.

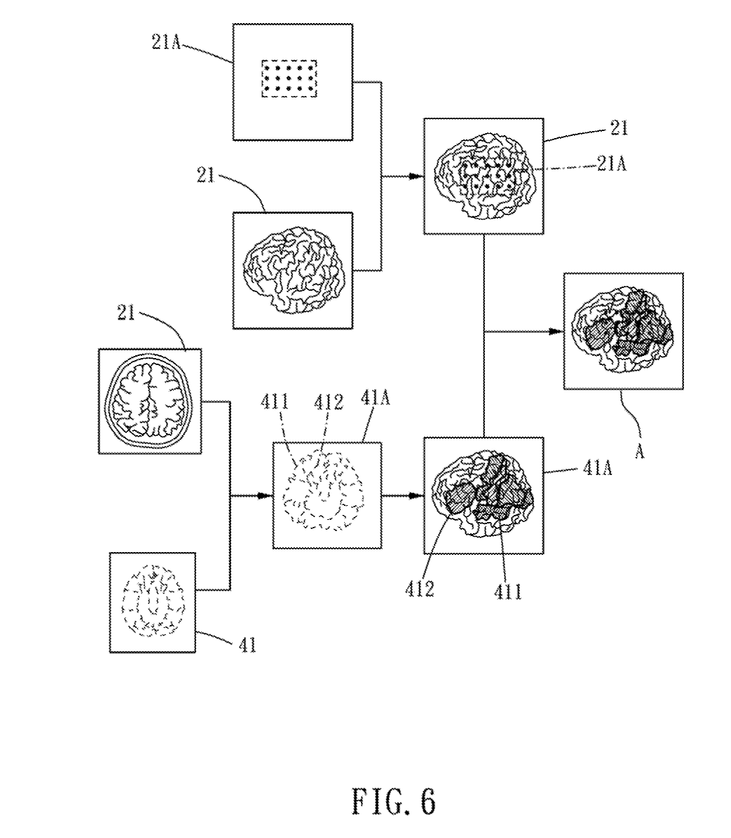

Regarding this brain functional map adjusting portion 40, it connects with the controller 30. The brain functional map adjusting portion 40 contains a brain functional map database 41 (which includes many original brain functional map cross-sectional images, such as 182 images or layers). The brain functional map adjusting portion 40 is able to obtain these 2D cross-sectional images 21 (assuming 182 images as well) and then to conduct a proportional deformation process so that the brain functional map database 41 will match with corresponding 2D cross-sectional images 21 (for the same number of images, such as 182). As a result, a plurality (such as 182) of two-dimensional (2D) adjusted brain functional map cross-sectional images 41A (see one shown in FIG. 6) can be obtained. Each 2D adjusted brain functional map cross-sectional image 41A contains a database brain profile line 411 and several database brain functional zones 412. During the proportional deformation process, each database brain profile line 411 is proportionally deformed to match with corresponding brain profile line 211 and the database brain functional zones 412 (inside the database brain profile line 411) are proportionally deformed to match with and fitted into the 2D cross-sectional images 21 so as to obtain a plurality of combined cross-sectional images A (see one illustrated in FIGS. 7 and 8) that can be transmitted to the controller 30.

The displaying portion 50 connects with the controller 30 for showing out these combined cross-sectional images A.

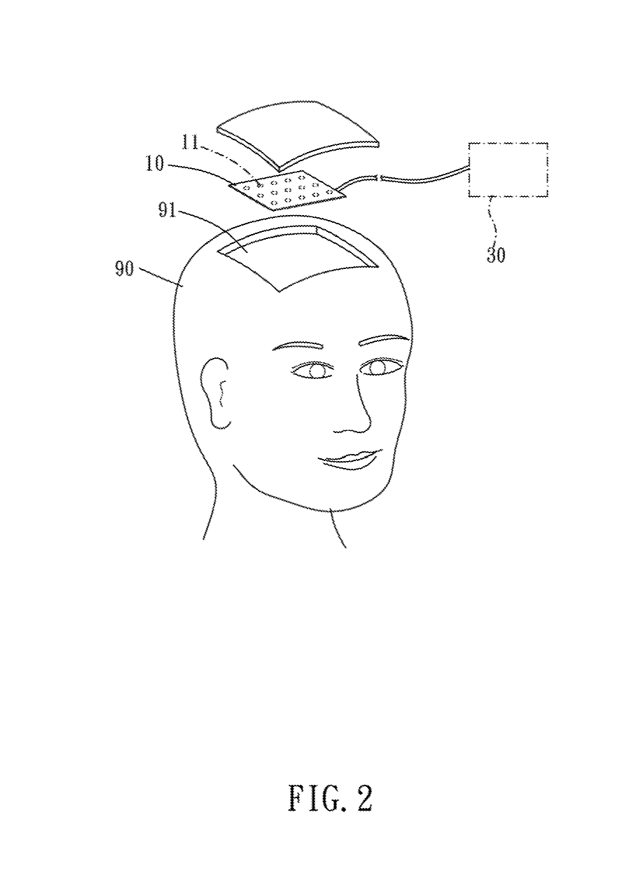

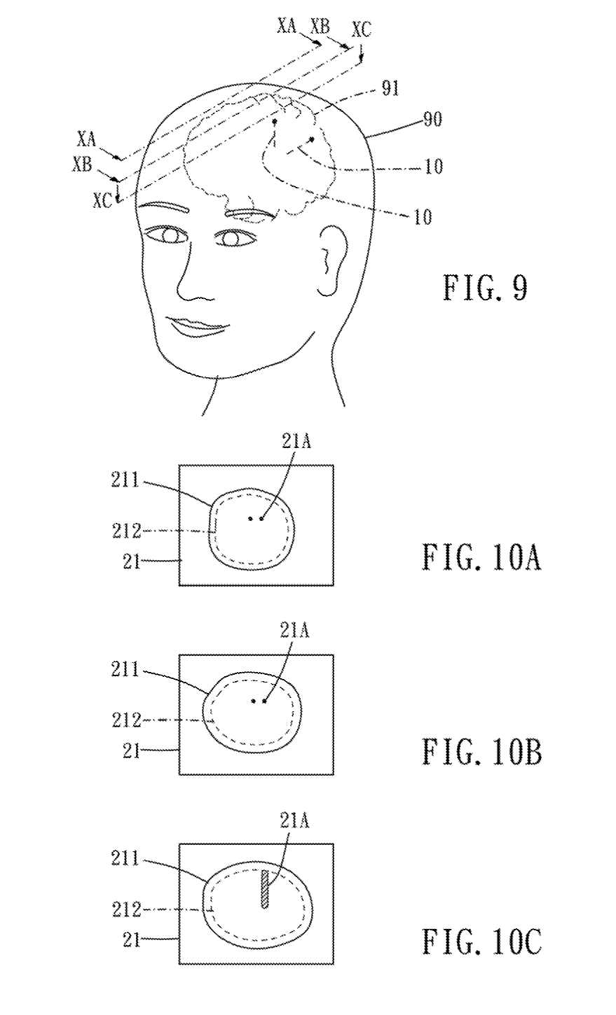

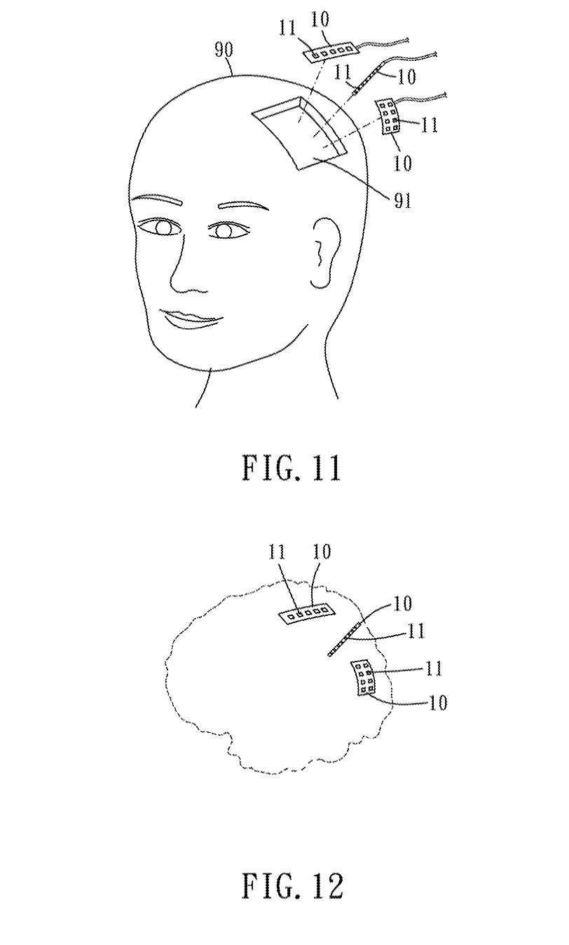

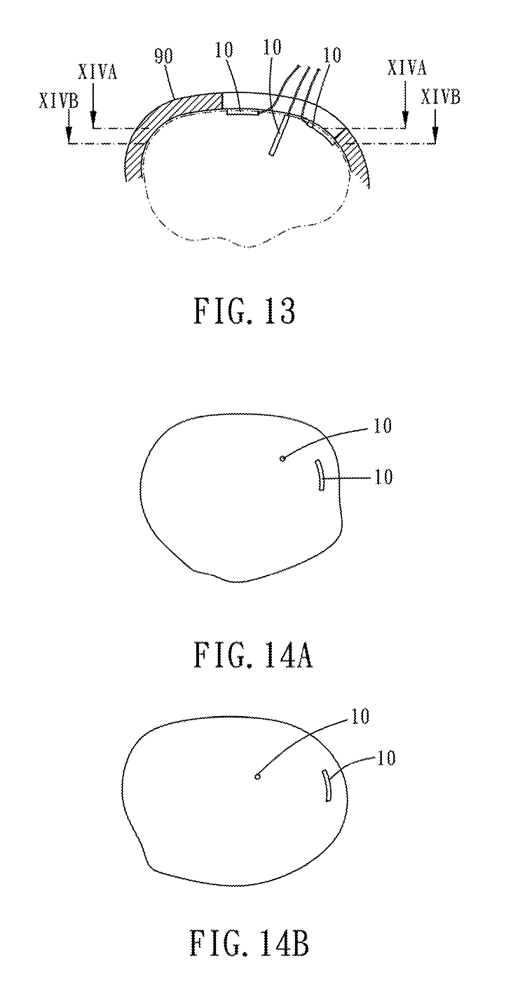

Practically, the electrode module 10 may be formed in different types or shapes.
[a] Thin-Film Type. As exhibited in FIG. 2, the electrode module 10 is formed as a thin film structure. Many electrodes 11 are disposed on this thin-film structure. It is very suitable to be placed on the cranial meninges.
[b] Needle Type. As shown in FIGS. 9, 10A, 10B and 10C. The electrode module 10 can be shaped like a needle structure. These electrodes 11 are just disposed on the needle structure so that it is easy to be inserted into a desired position inside the patient's brain.
[c] Mixed Type. Please see FIGS. 11, 12, 13, 14A and 14B. Under this condition, it contains both the structures of thin-film type and needle type as described above.

These electrodes 11 can detect the electrical waves (or variation) generated at several corresponding positions in the intracranial portion 91. Then, the position of the electrodes 11 can be shown in the 2D cross-sectional image(s) 21.
The image capturing module 20 can be one of the following devices, such as High-Resolution Magnetic Resonance Imaging (briefly referred as MRI) scanner, computed tomography (briefly referred as CT) scanner. Or course, it can be replaced by any other equivalent scanning or image capturing device as well.

The controller 30 can supply electricity to one or more specific positions in the intracranial portion 91 via these electrodes 11, so that it can effectuate the stimulation function.

Moreover, the afore-mentioned brain functional map database 41 can be selected from the commonly-used Brodmann brain atlas (briefly referred as BRODMANN), Automated Anatomical Labeling digital human brain atlas (briefly referred as AAL), or any other similar brain map database. If the BRODMANN is applied, the human brain is horizontally cut into 182 two dimensional images or layers. So, this three-dimensional scope (X, Y, Z coordinates information) about different functional zones can be obtained.

These electrodes 11 can detect the electrical waves at their corresponding positions. Besides, this invention can send out electricity to these corresponding positions via these electrodes 11 (in the reverse way). Both ways can be shown on the combined cross-sectional images A (combined by the 2D cross-sectional images 21 and the 2D adjusted brain functional map cross-sectional images 41A) via the display 50.

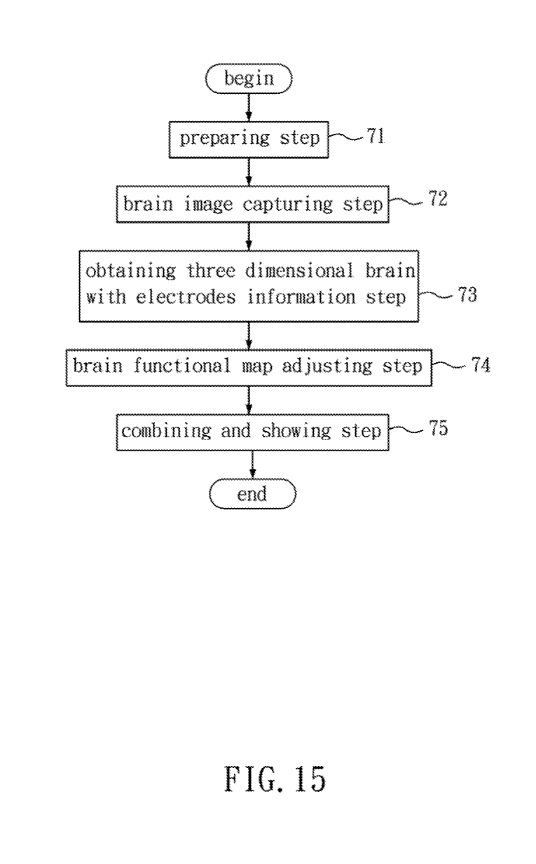

As illustrated in FIG. 15, it shows the displaying method of this invention.

The displaying method of personal brain structure displaying device having intracranial electrodes includes:
[1] Preparing Step 71: One can prepare an electrode module 10, an image capturing module 20, a controller 30, a brain functional map adjusting portion 40, and a displaying portion 50. About this electrode module 10, it is positioned inside an intracranial portion 91 of a human head 90. The electrode module 10 has multiple electrodes 11. The brain functional map adjusting portion 40 contains a brain functional map database 41.
[2] Brain Image Capturing Step 72: By using the image capturing module 20, one can capture brain images of this human head 90 in which these electrode modules 10 are already implanted.
[3] Obtaining Three-Dimensional Brain With Electrodes Information Step 73: The image capturing module 20 is able to obtain a three-dimensional (3D) brain information 20A which includes a plurality of two-dimensional (2D) cross-sectional images 21. Each 2D cross-sectional image 21 includes a brain profile line 211 and an inner brain area 212.
[4] Brain Functional Map Adjusting Step 74: The brain functional map adjusting portion 40 is able to obtain these 2D cross-sectional images 21 (assuming 182 images as well) and then to conduct a proportional deformation process so that the brain functional map database 41 (which includes many original brain functional map cross-sectional images, such as 182 images or layers) will match with corresponding 2D cross-sectional images 21 (for the same number of images, such as 182). As a result, a plurality (such as 182) of two-dimensional (2D) adjusted brain functional map cross-sectional images 41A (see one shown in FIG. 6) can be obtained. Each 2D adjusted brain functional map cross-sectional images 41A contains a database brain profile line 411 and several database brain functional zones 412. During the proportional deformation process, each database brain profile line 411 is proportionally deformed to match with corresponding brain profile line 211 and the database brain functional zones 412 (inside the database brain profile line 411) are proportionally deformed to match with and fitted into the 2D cross-sectional images 21 so as to obtain a plurality of combined cross-sectional images A (see one illustrated in FIGS. 7 and 8) that can be transmitted to the controller 30.
[5] Combining and Showing Step 75: the 2D adjusted brain functional map cross-sectional image 41A and the 2D cross-sectional image 21 are combined so as to obtain a plurality of combined cross-sectional images A.

Practically, the electrode module 10 may be formed in different types or shapes.
[a] Thin-Film Type. As exhibited in FIG. 2, the electrode module 10 is formed as a thin film structure. Many electrodes 11 are disposed on this thin-film structure. It is very suitable to be placed on the cranial meninges.
[b] Needle Type. As shown in FIGS. 9, 10A, 10B and 10C. The electrode module 10 can be shaped like a needle structure. These electrodes 11 are just disposed on the needle structure so that it is easy to be inserted into a desired position inside the patient's brain.
[c] Mixed Type. Please see FIGS. 11, 12, 13, 14A and 14B. Under this condition, it contains both the structures of thin-film type and needle type as described above.

These electrodes 11 can detect the electrical waves (or variation) generated at several corresponding positions in the intracranial portion 91. Then, the position of the electrodes 11 can be shown in the 2D cross-sectional image(s) 21.

The image capturing module 20 can be one of the following devices, such as High-Resolution Magnetic Resonance Imaging (briefly referred as MRI) scanner, computed tomography (briefly referred as CT) scanner. Or course, it can be replaced by any other equivalent scanning or image capturing device as well.

The controller 30 can supply electricity to one or more specific positions in the intracranial portion 91 via these electrodes, so that it can effectuate the stimulation function.

Moreover, the afore-mentioned brain functional map 41A can be selected from the commonly-used Brodmann brain atlas (briefly referred as BRODMANN), Automated Anatomical Labeling digital human brain atlas (briefly called as AAL), or any other similar brain map database. If the BORDMANN is applied, the human brain is horizontally cut into 182 two dimensional images or layers. So, the three-dimensional scope (X, Y, Z coordinates information) about different functional zones can be obtained.

These electrodes 11 can detect the electrical waves at their corresponding positions. Besides, this invention can send out electricity to these corresponding positions via these electrodes 11 (in the reverse way). Both ways can be shown on the combined cross-sectional images A (combined by the 2D cross-sectional images 21 and 2D adjusted brain functional map cross-sectional images 41A) via the display 50.

The advantages and functions are summarized as follows.
[1] The effects of intracranial electrodes are better than the traditional way. Because the electrodes are positioned inside the skull, all the collected electric signal and waves are more directly. Also, the one person applies external electricity to stimulate certain area of the brain inside the skull, it will be much better than the traditional one which is outside the skull. Hence, no matter the collection result or stimulation effect is much better than the traditional one.
[2] The brain structure information of a patient contains the precise positions of the electrodes and the corresponding brain functional areas. In this invention, the brain functional areas are fitted into the corresponding 2D cross-sectional image. So, the final combined cross-sectional images can be obtained. That is extremely helpful and convenient for the medical personnel to visualize the brain's functional areas as well as the precise position of the implanted electrodes. Furthermore, there is no need to rely on personal experience or prediction, especially for surgery. Thus, the brain structure information of a patient contains the precise positions of the electrodes and the corresponding brain functional areas
[3] The brain functional map can be adjusted and fitted into different patient's 2D cross-sectional images. The traditional brain functional map (or brain atlases) only divides the 3D structure of a person's brain into many functional areas (or zones). A doctor can refer to these functional areas to handle most patients with brain disease. However, the tradition brain functional map is based on the average result of certain number of persons. It is not suitable for all patients who have different brain sizes and shapes. But, this invention utilizes the adjusting and deformation techniques to adjust the existing brain functional map to fit into a specific person's brain. Therefore, this invention can use the traditional brain functional map to apply on the cross-sectional images of different patient.

The above embodiments are only used to illustrate the present invention, not intended to limit the scope thereof. Many modifications of the above embodiments can be made without departing from the claims of the present invention.

What is claimed is:

1. A personal brain structure displaying device having intracranial electrodes comprising:
an electrode module positioned inside a human head, said electrode module having multiple electrodes;
an image capturing module for capturing a brain area image of said human head, said image capturing module being able to obtain a three-dimensional (3D) brain information which includes a plurality of two-dimensional (2D) cross-sectional images; each 2D cross-sectional image including a brain profile line and an inner brain area; at least one 2D cross-sectional image containing an electrode image that is positioned on one of or both of said brain profile line and said inner brain area;
a controller connecting with said electrode module and said image capturing module for obtaining said three dimensional (3D) brain information;
a brain functional map adjusting portion connecting with said controller, said brain functional map adjusting portion containing a brain functional map database and being able to obtain said 2D cross-sectional images and then to conduct a proportional deformation process so that said brain functional map database matches with corresponding 2D cross-sectional images; a plurality of two-dimensional (2D) adjusted brain functional map cross-sectional images being obtained; each 2D adjusted brain functional map cross-sectional image containing a database brain profile line and several database brain functional zones; during said proportional deformation process, each database brain profile line being proportionally deformed to match with corresponding brain profile line and said brain functional zones being proportionally deformed accordingly to match with and fitted into said 2D cross-sectional images so as to obtain a plurality of combined cross-sectional images that can be transmitted to said controller; and
a displaying portion connecting with said controller for showing out these combined cross-sectional images.

2. The personal brain structure displaying device having intracranial electrodes as claimed in claim 1, wherein:
said electrode module is formed as a thin-film structure, a needle structure, or a combination of said thin-film structure and said needle structure; said electrodes are disposed on said thin-film structure, said needle structure, or said combination of said thin-film structure and said needle structure.

3. The personal brain structure displaying device having intracranial electrodes as claimed in claim 1, wherein:
said electrodes being able to detect electrical waves generated at several corresponding positions of the intracranial portion; then, the position of the electrodes being shown in the 2D cross-sectional image(s).

4. The personal brain structure displaying device having intracranial electrodes as claimed in claim 1, wherein:
said image capturing module being selected from a High-Resolution Magnetic Resonance Imaging (MRI) scanner or a computed tomography (CT) scanner.

5. The personal brain structure displaying device having intracranial electrodes as claimed in claim 1, wherein:
said controller being able to supply electricity to one or more specific positions in the intracranial portion via said electrodes for effectuating stimulation function.

6. A displaying method of a personal brain structure displaying device having intracranial electrodes comprising:
[1] A Preparing Step: to prepare an electrode module, an image capturing module, a controller, a brain functional map adjusting portion, and a displaying portion; said electrode module being positioned inside an intracranial portion of a human head; said electrode module having multiple electrodes; said brain functional map adjusting portion containing a brain functional map database;
[2] A Brain Image Capturing Step: by using said image capturing module to capture brain images of this human head in which these electrode modules are already implanted;
[3] An Obtaining Three-Dimensional Brain With Electrodes Information Step: said image capturing module being able to obtain a three-dimensional (3D) brain information which includes a plurality of two-dimensional (2D) cross-sectional images; each 2D cross-sectional image including a brain profile line and an inner brain area;
[4] A Brain Functional Map Adjusting Step: said brain functional map adjusting portion being able to obtain said 2D cross-sectional images and then to conduct a proportional deformation process so that said brain functional map database matches with corresponding 2D cross-sectional images; a plurality of two-dimensional (2D) adjusted brain functional map cross-sectional images being obtained; each 2D adjusted brain functional map cross-sectional image containing a database brain profile line and several database brain functional zones; during said proportional deformation process, each database brain profile line being proportionally deformed to match with corresponding brain profile line and said brain functional zones being proportionally deformed accordingly to match with and fitted into said 2D cross-sectional images so as to obtain a plurality of combined cross-sectional images that can be transmitted to said controller; and
[5] A Combining and Showing Step: said 2D adjusted brain functional map cross-sectional image and said 2D cross-sectional image being combined so as to obtain a plurality of combined cross-sectional images.

7. The displaying method of a personal brain structure displaying device having intracranial electrodes as claimed in claim 6, wherein:
said electrode module is formed as a thin-film structure, a needle structure, or a combination of said thin-film structure and said needle structure; said electrodes are disposed on said thin-film structure, said needle structure, or said combination of said thin-film structure and said needle structure.

8. The displaying method of a personal brain structure displaying device having intracranial electrodes as claimed in claim 6, wherein:
said electrodes being able to detect electrical waves generated at several corresponding positions of the intracranial portion; then, the position of the electrodes being shown in the 2D cross-sectional image(s).

9. The displaying method of a personal brain structure displaying device having intracranial electrodes as claimed in claim 6, wherein:
said controller being able to supply electricity to one or more specific positions in the intracranial portion via said electrodes for effectuating stimulation function.
